(19) United States
(12) Patent Application Publication
(10) Pub. No.: US 2015/0272691 A1
(43) Pub. Date: Oct. 1, 2015

(54) METHOD AND APPARATUS FOR PROVIDING VIRTUAL PLASTIC SURGERY SNS SERVICE

(71) Applicant: Tricubics Inc., San Jose, CA (US)

(72) Inventors: Jong Min KIM, San Jose, CA (US); Sang Ki LEE, San Jose, CA (US); Man Soo KIM, San Jose, CA (US); Min Ho AHN, San Jose, CA (US)

(73) Assignee: Tricubics Inc.

(21) Appl. No.: 14/478,428

(22) Filed: Sep. 5, 2014

(30) Foreign Application Priority Data
Mar. 31, 2014 (KR) 10-2014-0038190

(51) Int. Cl. A61B 19/00; G06T 17/00; G06T 11/00; A61B 5/00

(52) U.S. Cl. CPC A61B 19/50; A61B 5/0077; A61B 5/0013; A61B 5/0022; A61B 19/5212; G06T 17/00; G06T 11/001; A61B 2576/02; A61B 2019/505; A61B 2019/5295; G06T 2207/30201

(57) ABSTRACT

A virtual plastic surgery SNS service apparatus for providing a virtual plastic surgery service, comprising: a three-dimensional (3D) face model generation unit configured to generate a 3D face model by using an image from a portable camera; a 3D face model database management unit configured to store and manage the 3D face model and a result of virtual plastic surgery such that the 3D face model and the result of virtual plastic surgery are shared by people on a SNS (Social Network Service); a virtual plastic surgery simulation unit configured to allow a user to directly perform a simulation of virtual plastic surgery by using a certain facial part of a shared face model of a friend or by using a certain facial part of a known entertainer; and a social network service unit configured to allow the user to share the simulation result with people on the SNS or to connect the user to an expert to be consulted.

### CROSS-REFERENCE TO RELATED APPLICATIONS

This application is based on and claims priority from Korean Patent Application No. 10-2014-0038190, filed on Mar. 31, 2014, the disclosure of which is incorporated herein in its entirety by reference.

### TECHNICAL FIELD

The present disclosure relates to a method and apparatus for providing a virtual plastic surgery service; and, more particularly, to a service method and apparatus for creating a three-dimensional face model from a series of images captured by a portable camera such as a smart phone or a DSLR camera, sharing the face model with user's friends or experts, and performing a virtual plastic surgery simulation while allowing the user to share the simulation result with friends or to consult with experts.

### BACKGROUND

There already exists a service of sending information and face photo of a person (user) to an expert through a communications network to thereby allow the user to consult with the expert for an appearance that they will have after plastic surgery. Since, however, the consultation provided on-line is usually based on two-dimensional photos, it has been difficult for the user to be fully consulted on-line for the result of the plastic surgery.

To get three-dimensional (3D) virtual plastic surgery consultation to see changes before and after the plastic surgery, the user needs to visit a hospital specialized in plastic surgery and consult with experts there, which is troublesome and incurs expenses. Further, it has been practically impossible to consult with other experts in other hospitals for that result of the virtual plastic surgery consultation. Besides, from the point of view of the specialized hospital, since they need to purchase high-price equipment for generating the 3D face model, it takes high cost.

### SUMMARY

In view of the foregoing problems, the present disclosure provides a service method and apparatus for creating a 3D face model from a series of images captured by a portable camera such as a smart phone or a DSLR camera, sharing the face model with user's friends or experts, and performing a virtual plastic surgery simulation while allowing the user to share the simulation result with friends or to consult with experts.

However, the problems sought to be solved by the present disclosure are not limited to the above description and other problems can be clearly understood by those skilled in the art from the following description.

In accordance with an exemplary embodiment of the present disclosure, a virtual plastic surgery SNS service apparatus for providing a virtual plastic surgery service, comprising: a three-dimensional (3D) face model generation unit configured to generate a 3D face model by using an image from a portable camera; a 3D face model database management unit configured to store and manage the 3D face model and a result of virtual plastic surgery such that the 3D face model and the result of virtual plastic surgery are shared by people on a SNS (Social Network Service); a virtual plastic surgery simulation unit configured to allow a user to directly perform a simulation of virtual plastic surgery by using a certain facial part of a shared face model of a friend or by using a certain facial part of a known entertainer; and a social network service unit configured to allow the user to share the simulation result with people on the SNS or to connect the user to an expert to be consulted.

In accordance with another exemplary embodiment of the present disclosure, a method for providing a virtual plastic surgery service by using a virtual plastic surgery SNS service apparatus, the method comprising: generating a 3D face model by using an image from a portable camera; storing the generated 3D face model; setting an open range of the 3D face model of the user such that the 3D face model is shared with an expert in plastic surgery or a user's friend; consulting with an expert in plastic surgery for virtual plastic surgery by using the shared 3D face model; performing virtual plastic surgery in which the user replaces a certain part of the user with a corresponding part of a shared 3D face model of another person or a corresponding part of a 3D face model basically provided by the apparatus; and sharing a result of the virtual plastic surgery with friends on the social network service.

According to the above-described method and apparatus for providing the virtual plastic surgery service, the user is capable of modeling his/her 3D face easily at a tow price by using his/her own portable camera, and capable of consulting with an expert for the simulation result of virtual plastic surgery by sharing the 3D face model with the expert without needing to visit a hospital specialized in plastic surgery. Accordingly, it is possible to obtain a higher-quality simulation result for the virtual plastic surgery, as compared to conventional cases of obtaining two-dimensional face photos.

Further, according to the exemplary embodiments of the present disclosure, there is provided a service through which the user can share 3D face models with other people based on a social network service and is also capable of applying a certain facial part of the 3D face models to a corresponding part of his/her own 3D face model. Further, the user can show the simulation result to the other people including friends. Thus, the use of the virtual plastic surgery service may be facilitated.

Besides, according to the exemplary embodiments of the present disclosure, patients located far away from a hospital or patients staying overseas can be informed or check changes in their faces before and after plastic surgery, i.e., the result of plastic surgery before they visit the hospital. Thus, the quality of the medical service can be improved, and potential complains from the patients can be prevented. Thus, it is possible to provide an advantageous service for both the patients and the hospital

### BRIEF DESCRIPTION OF THE DRAWINGS

FIG. 1 is a diagram illustrating a virtual plastic surgery SNS service apparatus in accordance with an exemplary embodiment of the present disclosure.

FIG. 2 is a flowchart for describing a virtual plastic surgery SNS service process in accordance with an exemplary embodiment of the present disclosure.

FIG. 3 is a flowchart for describing a virtual plastic surgery simulation method for individual facial parts in accordance with an exemplary embodiment of the present disclosure.

### DETAILED DESCRIPTION

The advantages and features of the present disclosure and the ways to achieve them will become apparent from the following description of exemplary embodiments given in conjunction with the accompanying drawings. The exemplary embodiments will be described in detail so that inventive concept may be readily implemented by those skilled in the art.

However, it is to be noted that the exemplary embodiments are not intended to be anyway limiting and various modifications may be made without departing from the technical concept of the present disclosure. The scope of the inventive concept will be defined by the following claims rather than by the detailed description of the exemplary embodiments.

Through the whole document, the terms “the first” and the “the second” are used to designate various elements. However, the elements should not be limited by the terms, and the terms should be used only for the purpose of distinguishing one element from another. By way of example, without departing from the scope of the claims, the first element may be referred to as a second element, and, likewise, the second element may be referred to as a first element. Further, the term “and/or” is used to designate a combination of a plurality of related items or any one of these related items.

Through the whole document, the terms “connected to” or “coupled to” are used to designate a connection or coupling of one element to another element and include both a case where an element is directly connected or coupled to another element and a case where an element is indirectly connected or coupled to another element via still another element. Meanwhile, when the terms “directly connected to” or “directly coupled to” is used, it should be understood that there exists no other element between the two elements.

The various terms used in the present application are used to describe specific exemplary embodiments and are not meant to be anyway limiting. A singular form includes, unless otherwise defined in the context, a plural form. Through the whole document, the terms “include” or “have” are used to designate

Throughout the whole document, the term “comprises or includes” and/or “comprising or including” used in the document means that one or more other characteristics, numerical values, components, steps, operations, and/or the existence or addition of elements are not excluded in addition to the described components, steps, operations and/or elements.

Unless otherwise defined, all the terms including technical and scientific terminologies used in this document have the same meanings as those typically understood by those skilled in the art. The terms as defined in a generally used dictionary should be interpreted to have the same meanings as those understood in the context of the relevant art, and, unless defined clearly in the present document, should not be interpreted to have ideal or excessively formal meanings.

Hereinafter, exemplary embodiments of the present disclosure will be described in detail with reference to the accompanying drawings, which form a part hereof. Throughout the drawings and the following description, like parts will be assigned like reference numerals, and redundant description thereof will be omitted to facilitate understanding of the present disclosure.

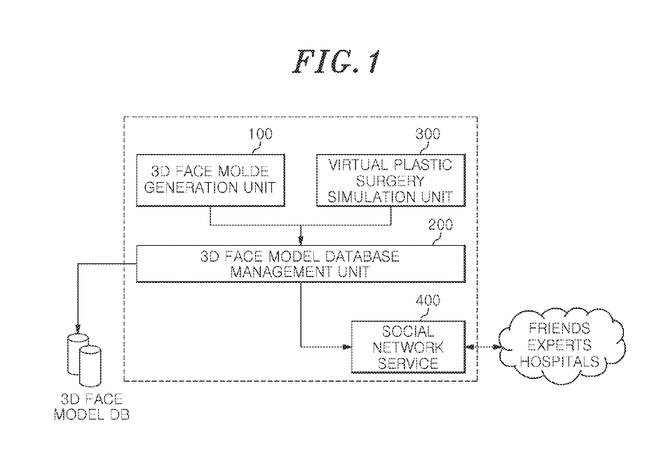

FIG. 1 is a diagram illustrating a virtual plastic surgery SNS service apparatus in accordance with an exemplary embodiment.

Referring to FIG. 1, the virtual plastic surgery SNS service apparatus of the exemplary embodiment includes a 3D face model generation unit 100, a 3D face model database management unit 200, a virtual plastic surgery simulation unit 300, and a social network service unit 400.

The 3D face model generation unit 100 is configured to receive images of a user's face inputted from a portable camera, analyze the series of face images of the user, generate 3D point clouds and then generate a 3D face mesh again. Here, the 3D face model generation unit 100 matches a 3D face standard model, which is modifiable part by part, to the 3D face mesh to thereby generate a 3D face model which can be modified part by part. At this time, in order to express user's face skin, the 3D face model generation unit 100 also generates a face skin texture map.

Thereafter, the 3D face model database management unit 200 stores the 3D face model and the skin texture map of the user in a 3D face model database along with a personal profile of the user, and manages the database. Then, the 3D face model database management unit 200 processes the 3D user face model such that the 3D face model stored therein can be used by the virtual plastic surgery simulation unit 300 and the social network service unit 400 later.

The social network service unit 400 sets an open range of 3D face model information of individual people on a social network through the 3D face model database management unit 200. For the open range of the 3D face model information of the individuals, a specific person such as an expert of a certain hospital or a user's friend, or a specific group such as a group of user's friends or family, a group of nose surgery specialists, or the like may be designated.

Thereafter, the virtual plastic surgery simulation unit 300 provides the user with a simulation in which certain parts of the 3D face model of the user can be modified by using 3D face model information opened to the user or by using templates provided by the system. Here, the modification may be made for individual facial parts. By way of example, the user can change his/her nose or eyes with a friend's nose or eyes. At this time, a boundary line of the modified part may be automatically corrected to fit to the user's face model.

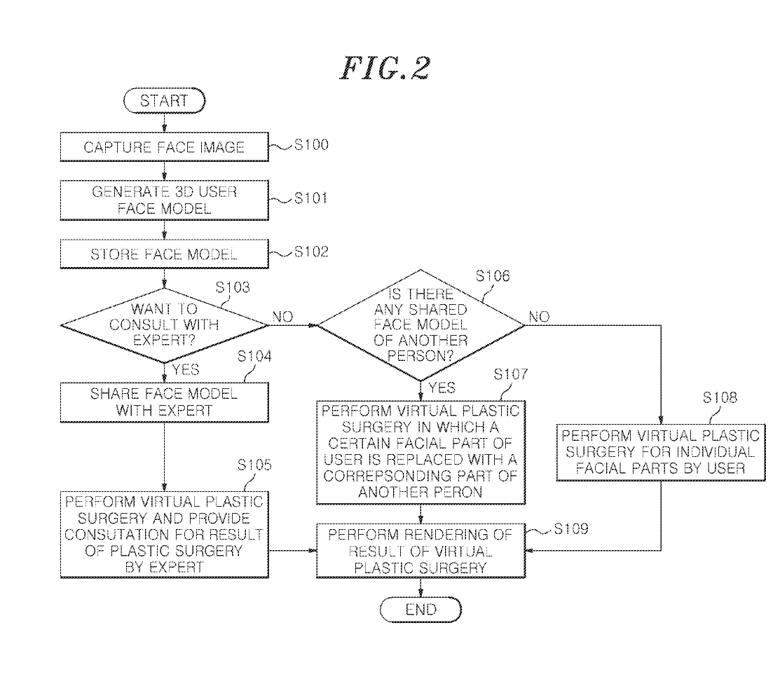

FIG. 2 is a flowchart for describing a virtual plastic surgery SNS service process in accordance with an exemplary embodiment of the present disclosure.

Referring to FIG. 2, face images of a user are captured from left to right or from right to left by a portable camera in accordance with an exemplary embodiment of the present disclosure (S100). The captured images may be moving pictures or a series of consecutive images.

Thereafter, a 3D user face model is generated by analyzing a correlation between the captured images (S101). In this process, 3D point clouds are generated first, and, then, a 3D face mesh is generated again. At this time, the 3D user face model which can be modified part by part is generated by matching a 3D face standard model, which is modifiable part by part, to the 3D face mesh.

At this time, a face skin texture map is also generated to express a face skin of the user. Thereafter, the mesh of the 3D face model and the face skin texture map are stored in the 3D face model database (S102). Thereafter, the user determines whether to consult with an expert for plastic surgery (S103). If the user wants to consult with the expert, the user opens his/her 3D face model to an expert or a group of experts (S104). Then, the expert performs virtual plastic surgery by using the 3D face model opened by the user and, then, sends a result of the virtual plastic surgery to the user who has requested that service (S105).

If, on the other hand, the user decides not o consult with an expert, the user checks whether there are 3D face models of other people that are shared (S106). If there are 3D face models of other people that are shared, the user may conduct a simulation of replacing a certain part of his/her face with a corresponding part of other people's face that are opened S107). If there are found no 3D face image of other people, the user may conduct a simulation of changing a certain part of his/her face by using templates for individual facial parts provided by the system (S108). At this time a boundary line of the modified part is automatically corrected to ft to the user's contour of face. Then, a result of the virtual plastic surgery is visually displayed on a monitor on the side of the user (S109).

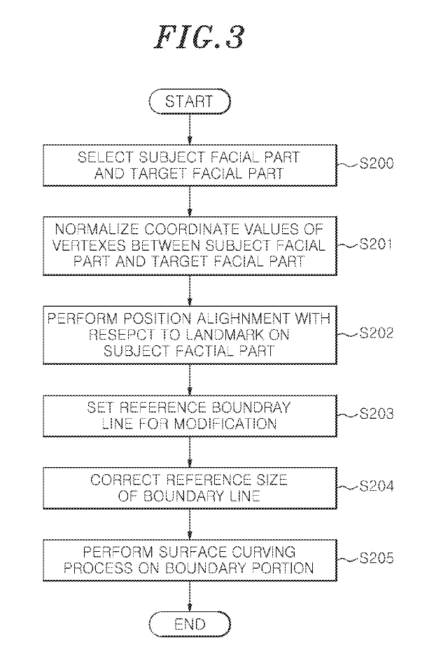

FIG. 3 is a flowchart for describing a virtual plastic surgery simulation method for individual facial parts in accordance with an exemplary embodiment of the present disclosure.

Referring to FIG. 3, a subject facial part, such as a nose, an eye or a chin, of a to be subjected to virtual plastic surgery is selected from a user's face, and a target facial part with which the subject facial part of the user is to be replaced is also selected (S200). Since coordinate values of 3D vertexes forming the subject facial part and the target facial part may be different, normalization is performed to adjust them (S201).

Subsequently, a mesh of the target facial part is aligned with respect to a landmark on the subject facial part (S202), Here, the landmark may be defined as certain points that are not changed during the plastic surgery process.

Thereafter, it is determined whether matching of the subject facial part and the target facial part is to be performed with respect to a boundary line of the subject facial part, with respect to a boundary line of the target facial part, or with respect to an intermediate value between the two boundary lines (S203).

Then, with respect to the set boundary line, the mesh of the subject facial part is eliminated, and the mesh of the target facial part is synthesized thereto (S204). At this time, automatic correction may be made to align the boundary lines. Thereafter, since the curved contour of the user's face and the synthesized boundary portion may not be smooth, there is performed a surface curving process of smoothening boundary lines of surfaces by changing normal vectors between the two connected meshes (S205).

Although exemplary embodiments of the present disclosure are described above with reference to the accompanying drawings, those skilled in the art will understand that the present disclosure may be implemented in various ways without changing the necessary features or the spirit of the present disclosure. Therefore, it should be understood that the exemplary embodiments described above are not limiting, but only an example in all respects. The scope of the present disclosure is expressed by claims below, not the detailed description, and it should be construed that all changes and modifications achieved from the meanings and scope of claims and equivalent concepts are included in the scope of the present disclosure.

### EXPLANATION OF REFERENCE NUMERALS

100: 3D face model generation unit

200: 3D face model database management unit

300: Virtual plastic surgery simulation unit

400: Social network service unit

What is claimed is:

1. A virtual plastic surgery SNS service apparatus for providing a virtual plastic surgery service, comprising:
a three-dimensional (3D) face model generation unit configured to generate a 3D face model by using an image from a portable camera;
a 3D face model database management unit configured to store and manage the 3D face model and a result of virtual plastic surgery such that the 3D face model and the result of virtual plastic surgery are shared by people on a SNS (Social Network Service);
a virtual plastic surgery simulation unit configured to allow a user to directly perform a simulation of virtual plastic surgery by using a certain facial part of a shared face model of another person on the SNS; and
a social network service unit configured to allow the user to share the simulation result with people on the SNS or to connect the user to an expert to be consulted.

2. The virtual plastic surgery SNS service apparatus of claim 1,
wherein the 3D face model generation unit is configured to receive Rice images of the user from the portable camera, generate 3D point clouds by analyzing a series of face images of the user, and generate a 3D face mesh and a skin texture map.

3. The virtual plastic surgery SNS service apparatus of claim 1,
wherein the 3D face model generation unit s configured to generate the 3D face model, which is modifiable part by part, by matching a 3D face standard model, which is modifiable part by part, to the 3D face mesh.

4. The virtual plastic surgery SNS service apparatus of claim 1,
wherein the social network service unit is configured to designate a certain person or a certain group of people as an open range of a 3D face model of an individual person stored in a 3D user face database.

5. The virtual plastic surgery SNS service apparatus of claim 1,
wherein the virtual plastic surgery simulation unit s configured to provide a simulation of modifying a certain part of the 3D face model of the user by using 3D face model information opened to the user or by using a template provided by a system.

6. The virtual plastic surgery SNS service apparatus of claim 1,
wherein virtual plastic surgery is performed for individual facial parts in the virtual plastic surgery simulation unit, and, at this time, a boundary line of a modified part is automatically corrected to fit to the face model of the user.

7. A method for providing a virtual plastic surgery service by using a virtual plastic surgery SNS service apparatus, the method comprising:
generating a 3D face model by using an image from a portable camera;
storing the generated 3D face model;
setting an open range of the 3D face model of the user such that the 3D face model is shared with an expert in plastic surgery or people on a social network service;
consulting with an expert in plastic surgery for virtual plastic surgery by using the shared 3D face model;
performing virtual plastic surgery in which the user replaces a certain part of the user with a corresponding part of a shared 3D face model of another person or a corresponding part of a 3D face model basically provided by the apparatus; and
sharing a result of the virtual plastic surgery with people on the social network service.

8. The method of claim 7,
wherein the process of generating the 3D face model by using the image from the portable camera includes:
receiving moving pictures or consecutive images;
generating 3D point clouds by analyzing a correlation between he images;
generating a 3D face mesh;
generating a 3D face model, which is modifiable part by part, by matching a 3D face standard model, which is modifiable part by part, to the 3D face mesh; and
generating a face skin texture map for expressing a face skin of the user.

9. The method of claim 7,
wherein the process of setting the open range of the 3D face model of the user such that the 3D face model is shared by the expert or the people on the social network service includes:
setting the 3D face model of the user to be shared with a friend or a group of friends or to be shared with an expert or a group of experts for the user o be consulted for plastic surgery.

10. The method of claim 7,
wherein the process of consulting with the expert in plastic surgery for virtual plastic surgery by using the shared 3D face model includes a process in which the expert performs a virtual plastic surgery simulation by using the shared 3D face model of the user and provides the user with a result showing his/her appearances after plastic surgery.

11. The method of claim 7,
wherein the process of performing virtual plastic surgery in which the user replaces the certain part of the user with the corresponding part of the shared 3D face model of another person or the corresponding part of the 3D face model basically provided by the apparatus includes:
checking whether there is any shared 3D face model of another person;
if there is any shared 3D face model of another person, performing a simulation of replacing the certain part of the user face with the corresponding facial part of that person; and
if there is found no shared 3D face mode of another person, performing a. simulation of replacing the certain part of the user sing templates for respective facial parts provided by a system.

12. The method of claim 7,
wherein the process of performing virtual plastic surgery in which the user replaces the certain part of the user with the corresponding part of the shared 3D face model of another person or the corresponding part of the 3D face model basically provided by the apparatus includes:
selecting a subject facial part of the user to be subjected to virtual plastic surgery and a target facial part with which the subject racial part is to be replaced;
normalizing coordinate values of 3D vertexes of meshes forming the subject facial part and the target facial part;
aligning the mesh of the target facial part with respect to a landmark on the subject facial part;
matching boundary lines between the subject facial part and the target facial part; and
performing a surface curving process for the boundary line.
